(19) United States
(12) Patent Application Publication
(10) Pub. No.: US 2026/0055451 A1
(43) Pub. Date: Feb. 26, 2026

(54) SYSTEM, APPARATUS AND PROCESS

(71) Applicant: Micromole Inc., Berkeley, CA (US)

(72) Inventors: Ethan Samuel Volavola Vidal, Turner (AU); Ella Chernis, Almaty (KZ)

(21) Appl. No.: 19/103,641

(22) PCT Filed: Aug. 14, 2023
(86) PCT No.: PCT/AU2023/050765

§ 371 Date: Feb. 13, 2025

(30) Foreign Application Priority Data
Aug. 15, 2022 (AU) 2022902309
Mar. 31, 2023 (AU) 2023202006

(51) Int. Cl. C12Q 1/6869; G01N 27/327

(52) U.S. Cl. CPC C12Q 1/6869; G01N 27/3278

(57) ABSTRACT

A system for nucleotide chain sequencing, the system including an apparatus with: at least one electrode for measuring a collective electromagnetic signal from a portion of a nucleotide chain, the portion including a plurality of nucleotides; and a mechanism configured to move the nucleotide chain relative to the electrode in order to modulate the collective electromagnetic signal, for identifying an electromagnetic signal associated with each individual nucleotide in the plurality of nucleotides.

### RELATED APPLICATIONS

The present application relates to Australian Provisional Patent Application No. 2022902309, and Australian Complete Patent Application No. 2023202006, the originally filed specifications of which, and the amended specifications of which, are hereby incorporated by reference herein.

### TECHNICAL FIELD

The present disclosure relates to systems, apparatuses and processes for nucleotide chain sequencing, including sequencing of nucleic chains, deoxyribonucleic acid (DNA), single strand (ssDNA), and/or molecules including ribonucleic acid (RNA).

### BACKGROUND

Nucleic acid sequencing includes determining the order of nucleotides in a nucleotide chain, which is typically a nucleic acid molecule—which can be DNA, ssDNA, RNA and/or proteins/molecules including RNA, including determining the order of bases in the nucleotide chain, which include: adenine (A), guanine (G), cytosine (C), and thymine (T).

Rapid nucleotide sequencing has greatly accelerated biological and medical research and discovery.

However, existing systems and processes for nucleotide sequencing may be insufficiently rapid/accurate/efficient for at least some applications.

It is desired to address or ameliorate one or more disadvantages or limitations associated with the prior art, or to at least provide a useful alternative.

### SUMMARY

According to the present invention, there is provided a system for nucleotide chain sequencing, the system including an apparatus with:
at least one electrode for measuring a collective electromagnetic signal from a portion of a nucleotide chain, the portion including a plurality of nucleotides; and
a mechanism configured to move the nucleotide chain relative to the electrode in order to modulate the collective electromagnetic signal, for identifying an electromagnetic signal associated with each individual nucleotide in the plurality of nucleotides.

According to the present invention, there is also provided a process for nucleotide chain sequencing including:
measuring collective electromagnetic signals from a portion of a nucleotide chain using at least one electrode, the portion of the nucleotide chain including a plurality of nucleotides; and
moving the nucleotide chain relative to the at least one electrode in order to modulate the collective electromagnetic signals, for identifying an electromagnetic signal associated with each individual nucleotide in the plurality of nucleotides.

### BRIEF DESCRIPTION OF THE DRAWINGS

Some embodiments of the present invention are hereinafter described with reference to the accompanying drawings in which:

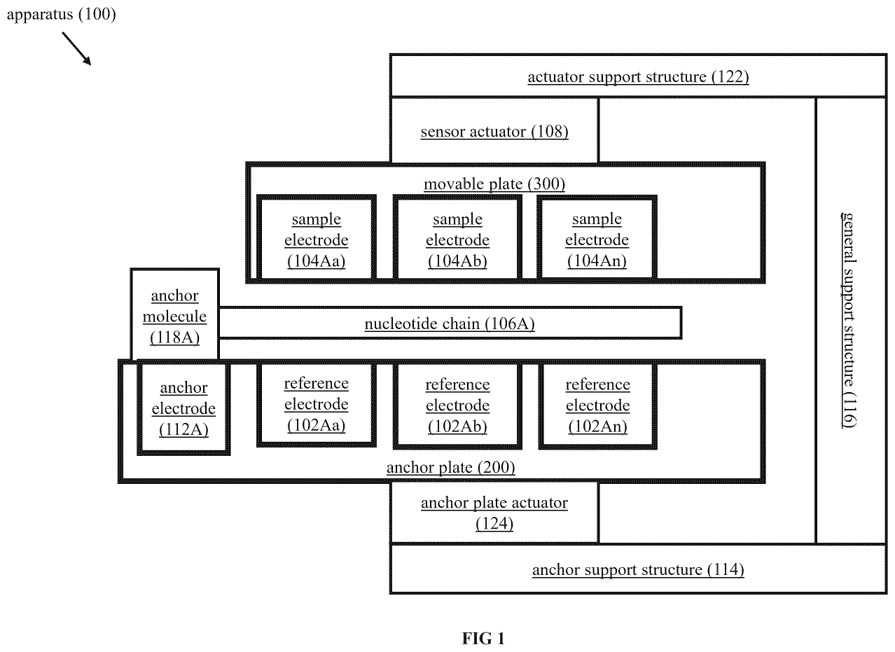

a) FIG. 1 is a schematic diagram of a right side cross-sectional view of an apparatus (“sequencer apparatus”) for nucleotide chain sequencing according to a first embodiment of the present disclosure;

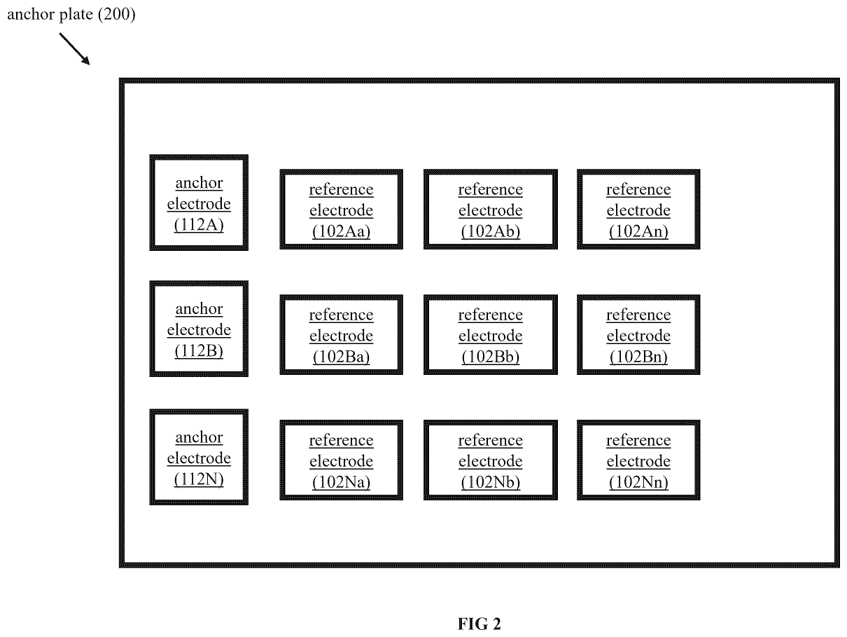

b) FIG. 2 is a schematic diagram of a top side view of an anchor plate of the apparatus according to the first embodiment of the present disclosure;

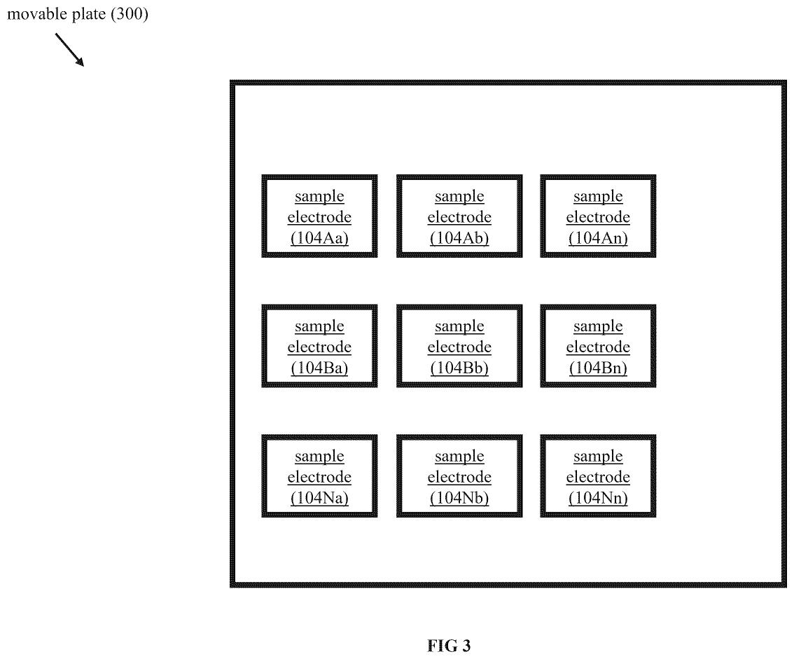

c) FIG. 3 is a schematic diagram of an underside view of a movable plate of the apparatus according to the first embodiment of the present disclosure;

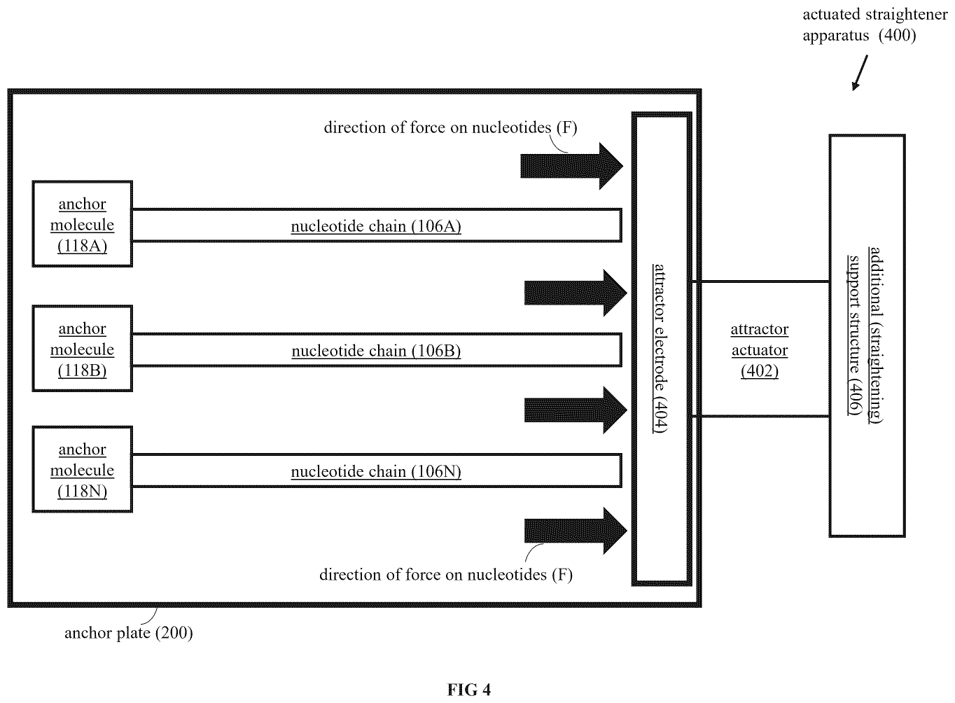

d) FIG. 4 is a schematic diagram of a top side view of an actuated straightener apparatus for nucleotide chain straightening (in solution) according to the first embodiment of the present disclosure;

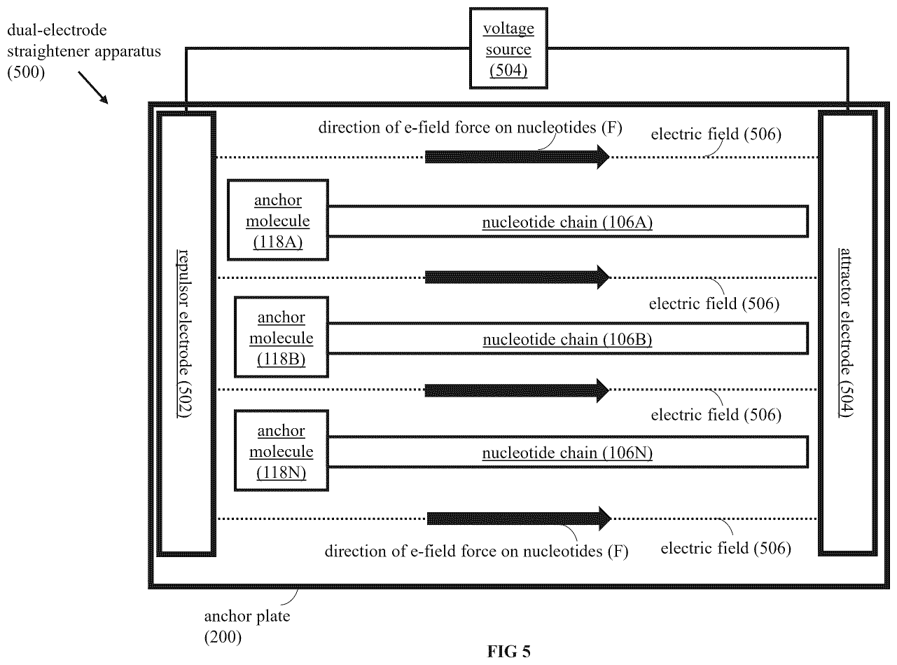

e) FIG. 5 is a schematic diagram of a top side view of a dual-electrode straightener apparatus for nucleotide chain straightening according to the first embodiment of the present disclosure;

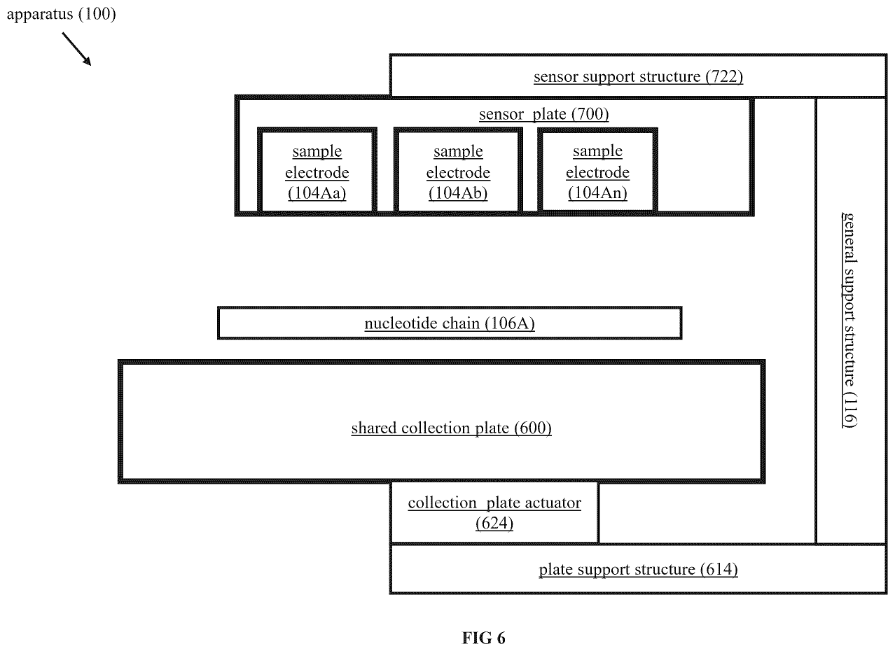

f) FIG. 6 is a schematic diagram of a right side cross-sectional view of an apparatus (“sequencer apparatus”) for nucleotide chain sequencing according to a second embodiment of the present disclosure;

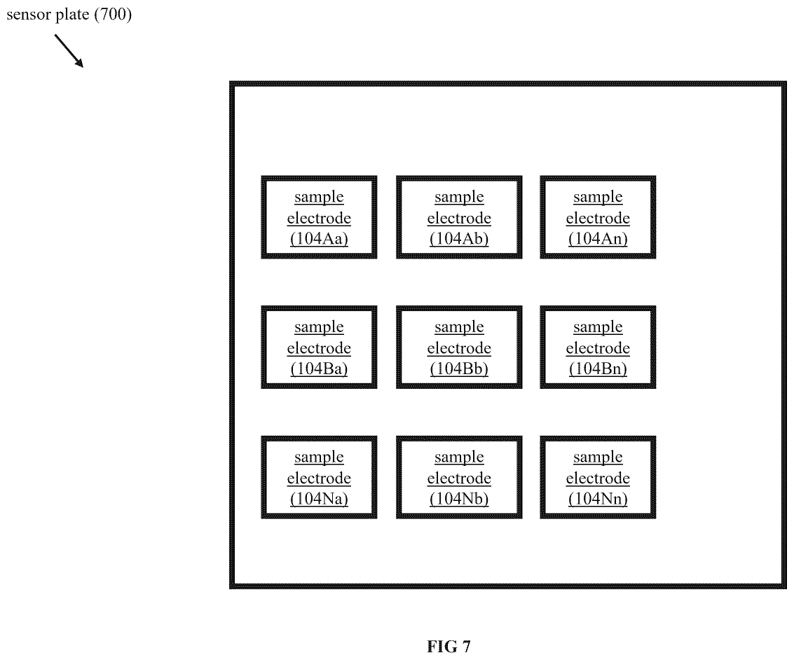

g) FIG. 7 is a schematic diagram of an underside view of a movable plate of the apparatus according to the second embodiment of the present disclosure; and

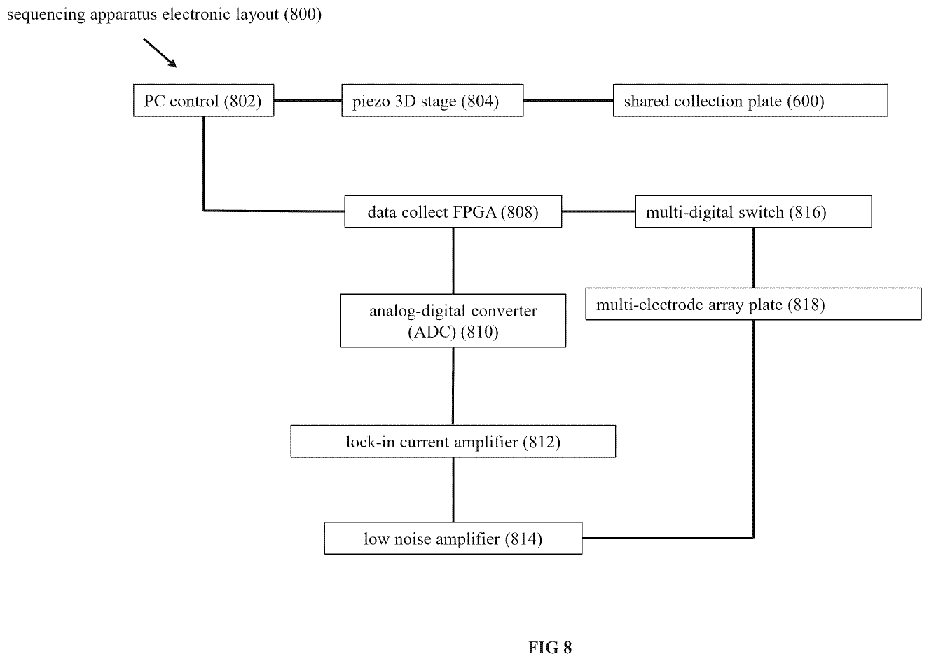

h) FIG. 8 is a schematic diagram of an exemplary electronic system of the apparatus according to the second embodiment of the present disclosure.

### DETAILED DESCRIPTION

### I. System and Sequencing Apparatus

Described herein is a system for nucleotide chain sequencing.

The system includes an apparatus (100) (“sequencing apparatus”, “sequencer” or “sequencer apparatus”) for nucleotide chain sequencing.

The apparatus (100) has at least one electrode (“sample electrode” or “probe”) for measuring an electromagnetic signal (e.g., an electrical charge, an electrical current and/or a voltage) from a portion of a nucleotide chain.

The apparatus (100) further has a mechanism configured to move the nucleotide chain relative to the electrode, in order to modulate the electromagnetic signal.

### II. First Embodiment

A first embodiment of the system including the apparatus 100 is described hereinafter.

### General Structure of the Sequencing Apparatus

As shown in FIG. 1, the sequencer apparatus (100) according to this embodiment includes:
a) at least one electromagnetic (EM) sample electrode (104A, including 104Aa, 104Ab, . . . , 104An) for measuring an EM signal (“sample EM signal”) (e.g., an electrical charge, an electrical current or a voltage) from a portion (or “sample portion”) of a nucleotide chain (106A); and
b) a mechanism (or “modulation mechanism”) configured to move the nucleotide chain (106A) relative to the EM sample electrode (104A), in order to modulate the EM signal.

The at least one EM sample electrode (104A) may be a non-contact EM sensor. That is, it does not contact or touch the nucleotide chain (106A) during the measuring process.

The sequencer apparatus (100) may include at least one EM reference electrode (102A, including 102Aa, 102Ab, . . . , 102An) configured to generate or measure an EM signal (“reference EM signal”) (e.g., an electrical charge and/or an electrical current) from a portion (or “reference portion”) of the nucleotide chain (106A) that is adjacent each respective the reference electrode (102A). The one or more reference electrodes (102A) may be controlled, as described hereinafter, to substantially essentially sandwich the nucleotide chain (106A) with nm precision in order to acquire highly reliable electrical signals with an acceptable signal to noise ratio (SNR).

The EM signals measured may include one or more of the following:
a) Variations in electrical current (conductance) between a reference electrode (102X) and a sample electrode (104X), caused by the effect of different local charge densities for different nucleotides, which may create dipole moments or modify the potential barrier for quantum tunnelling of the electrons. These variations may naturally be constant (DC), at determined frequencies (AC) due to particular molecular resonances, or cover broader parts of the frequency spectrum.
b) Variations in electrical current conducted through part of the nucleotide chain (106A), from the anchor electrode (112X) to one of the sample electrodes (104Xx).
c) Reference electrical current conducted through part of the nucleotide chain (106A), from the anchor electrode (112X) to one of the reference electrodes (102Xx). This signal may be monitored to adjust/correct the signal from (b) to external noise sources, thermal drift, or Brownian motion.
d) Variations in electrical current as in (a) and (b), relative to a fixed-frequency modulation applied to the anchor plate (200) via the plate actuator (124).

The modulation mechanism includes:
a) a holder configured to hold/secure the nucleotide chain (106) such that the nucleotide chain (106) is stationary relative to an anchor portion of the sequencer apparatus (100); and
b) at least one controlled actuator, including a sensor actuator (108) and/or an anchor plate actuator (124), configured to move a sensor portion of the sequencer apparatus (100) relative to the anchor portion, wherein the sensor portion includes the at least one EM sample electrode (104A).

The anchor portion includes:
a) an anchor plate (200) with:
   i) an array of the one or more reference electrodes (102Aa, 102Ab, . . . , 102An), and
   ii) the at least one anchor electrode (112A) configured to hold/secure the nucleotide chain (106A) to the anchor portion (and to conduct the electrical current along the nucleotide chain (106A) as described hereinbefore);
b) the anchor plate actuator (124); and
c) an anchor support (114) that connects the anchor plate (200), including via the anchor plate actuator (124), to a general support structure (116), thereby supporting the anchor plate (200) during a measurement process of the sequencer apparatus (100).

The holder includes:
a) the anchor electrode (112A);
b) an anchor molecule (118A) configured/selected to hold/secure the nucleotide chain (106A) to its corresponding anchor electrode (112A)—the anchor molecule (118A) may include a self-assembled monolayer (SAM) for DNA surface immobilization, e.g., as described in Cynthia Bamdad's paper entitled “A DNA Self-Assembled Monolayer for the Specific Attachment of Unmodified Double-or Single-Stranded DNA” in the Biophysical Journal (V. 75, Oct. 1998, pp. 1997-2003); and/or
c) the one or more reference electrodes (102Xx), which may be configured to attract/hold/secure the nucleotide chains (106X) to the anchor plate (200) to assist with holding the nucleotide chains (106X) stationary relative to the anchor plate (200) as the movable plate (300) is moved relative thereto to generated the EM signal that is detected.

The sensor portion includes:
a) a movable plate (300) with an array of one or more of the sample electrodes (104Aa, 104Ab, . . . , 104An); and
b) the sensor actuator (108) configured to movably hold/secure the movable plate (300) to an actuator support structure (122) that fixes the movable plate (300) in controllably movable relation to the general support structure (116).

As shown in FIG. 2, the anchor plate (200) includes one or more parallel linear arrays of the two or more reference electrodes (102Aa, 102Ab, . . . , 102An), thus forming a two-dimensional (2D) array of the reference electrodes (102Aa, 102Ab, . . . , 102An; 102Ba, 102Bb, . . . , 102Bn; 102Na, 102Nb, . . . , 102Nn). The anchor plate (200) includes one or more of the at least one anchor electrode (112A, 112B, . . . , 112N), and there is one anchor electrode (112) for each linear array of the reference electrodes (102a, 102b, . . . , 102n) because each anchor electrode (112) holds one nucleotide chain (106) in position over/along the corresponding linear array of the reference electrodes (102a, 102b, . . . , 102n). Alternatively, the anchor plate (200) may include the reference electrode plate that corresponds to two or more of the sample electrodes (104Aa, 104Ab, . . . , 104An), thus has a width and/or a length corresponding to two or more times the width and/or the length of each of the corresponding sample electrodes (104Aa, 104Ab, . . . , 104An). The reference electrode plate may receive the nucleotide chains (106X) in use. The reference electrode chip may be at least 1%, 5% or 10% bigger in surface area than a surface area of the array of corresponding sample electrodes to facilitate handling and/or for placing larger amounts of DNA samples.

Optionally, the sensor portion of the sequencer apparatus (100) may include an electrical insulator between each of the reference electrodes (102Xx) and where the nucleotide chains (106X) are held/secured in position during the measurement process, e.g., at least one insulator layer on an outer surface of each reference electrode (102Xx).

As shown in FIG. 3, the movable plate (300) includes one or more parallel linear arrays of the one or more sample electrodes (104Aa, 104Ab, . . . , 104An), thus forming a 2D array of the sample electrodes (104Aa, 104Ab, . . . , 104An; 104Ba, 104Bb, . . . , 104Bn; 104Na, 104Nb, . . . , 104Nn).

The anchor portion of the sequencer apparatus (100) includes an electrical insulator between each of the sample electrodes (104Xx) and where the nucleotide chains (106X) are held/secured in position during the measurement process, e.g., at least one insulator layer on an outer surface of each sample electrode (104Xx).

### Straightening Apparatuses

The system includes at least one apparatus (“straightening apparatus”) for straightening the nucleotide chains (106A, 106B, . . . , 106N) before insertion into the sequencer apparatus (100). The straightening apparatus may include:
a) an actuated nucleotide chain attractor for nucleotide chain straightening in solution, as shown in FIG. 4; and/or
b) a dual-electrode nucleotide chain attractor for nucleotide chain straightening in solution, as shown in FIG. 5.

During the straightening, as shown in FIG. 4 and FIG. 5, the one or more nucleotide chains (106A, 106B, . . . , 106N) are held/secured to the respective anchor molecules (118A, 118B, . . . , 118N), and straightened/stretched/extended/unwound over/along the corresponding linear array of the reference electrodes (102Xa, 102Xa, . . . , 102Xn—where “X” is “A, B, . . . , N”) by an attractor electrode (404, 504) that applies an attractive force to the nucleotide chains (106A, 106B, . . . , 106N) in order to straighten/stretch/unwind the nucleotide chains (106X). The attractive force is directed away from the anchor molecules (118A, 118B, . . . , 118N) and along each linear array of the reference electrodes (102Xa, 102Xa, . . . , 102Xn). The reference electrodes (102Xx) may be controlled to assist in the straightening/stretching/extending/unwinding of the nucleotide chains (106X) by applying an electronic charge to the reference electrodes (102Xx) to attract the nucleotide chains (106X) over/along the linear array of the reference electrodes (102Xx).

As shown in FIG. 4, one form of the attractor electrode (404) may be included in an actuated straightener apparatus (400) that includes:
a) the attractor electrode (404); and
b) an actuator (“attractor actuator”) (402) that is mechanically coupled to the attractor electrode (404) and an additional support structure (406) such that the attractor electrode (404) is movable (by the attractor actuator (402)) from a position close to the anchor molecules (118X) to a position further from the anchor molecules (118X), after the nucleotide chains (106X) have been held/secured thereto, in order to draw/pull the nucleotide chains (106X) and straighten/stretch/extend them over/along the corresponding linear arrays of the reference electrodes (102Xx—where “x” is “a, b, . . . , n”).

As shown in FIG. 5, one form of the attractor electrode (504) may be included in the dual-electrode straightener apparatus (500) that includes:
a) the attractor electrode (504); and
b) a repulsor electrode (502) that is arranged to be biased by a voltage source (508) to form an electric field (506) that surrounds the nucleotide chains (106X)—the voltage source (508) may pulse/modulate the electric field (506) to progressively unwind the nucleotide chains (106X) during a preparation time prior to the sensing of the EM signals.

As shown in FIG. 4 and FIG. 5, the attractor electrode (404,504) applies a force (F) to the nucleotide chains (106X) in a direction away from the anchor molecules (118X) and along the linear arrays of the reference electrodes (102Xx). As the linear arrays of the reference electrodes (102Xx) are aligned and collinear with the respective linear arrays of the sample electrodes (104Xx), the nucleotide chains (106X) are also straightened/stretched/extended/positioned under/along the corresponding linear array of the sample electrodes (104Xx) by the force (F) from the anchor molecules (118X) and the attractor electrode (404,504).

The dual-electrode straightener apparatus (500) and/or the actuated straightener apparatus (400) may include an electrolyte solution in which the nucleotide chains (106X) are immersed. The electrolyte solution is selected to at least partially denature the nucleotide chains (106X), thus allowing them to be straightened/stretched/extended by the force (F).

The straightener apparatus (400,500) may include a tray/bath to hold the electrolyte solution around the nucleotide chains (106X).

The straightener apparatus (400,500) may include a heater, including with/in the tray/bath, to heat the nucleotide chains (106X) such that they are denatured, e.g., to substantially 90 degrees C, in a fluid and/or the electrolyte solution.

Alternatively, the nucleotide chains (106X) may be secured to the anchor plate (200) by using other suitable methods/techniques that can hold the nucleotide chains (106X) in place, e.g., surface treatment of the anchor plate (200) (e.g., hydrophobicity), allocating some of the reference electrodes (102Xx) as ‘nodes’ to clamp the nucleotide chains (106X) in place with electrostatic charges (therefore not using those reference electrodes for measurement), or cryogenic cooling of the nucleotide chains (106X) together with the anchor plate (200).

### Actuators and Alignment

The sensor actuator (108), the anchor plate actuator (124), and the attractor actuator (402) may include piezo-electric actuators, e.g., from PI (Physik Instrumente), or piezoceramic actuators.

The anchor portion may include a stepper motor, which is configured to move the anchor plate (200) into alignment with the movable plate (300)—including in all 3 orthogonal directions, X, Y, Z, and additionally/optionally three rotational dimensions (e.g. Euler angles α, β, γ)—after the nucleotide chain (106) has been secured in place on/along the anchor plate (200). The sensor portion may include a stepper motor, which is configured with the sensor actuator (108) to also move the anchor plate (200) into alignment with the movable plate (300)—including in all 3 orthogonal directions, X, Y, Z, and additionally/optionally three rotational dimensions (e.g. Euler angles α, β, γ)—after the nucleotide chain (106) has been secured in place on/along the anchor plate (200). The anchor plate (200) and the movable plate (300) are thus moved into alignment (or “position” or “operational condition”) after the nucleotide chains (106X) have been anchored and straightened in place: in this alignment/operational condition, there is a small gap between the plates (200,300) and they are substantially plane parallel, e.g., the small gap may be less than 1 micrometer, less than 100 nanometers (nm), or less than 10 nm, e.g., of the order of a width of a DNA molecule (substantially 0.3 nm to 2.5 nm), but substantially more than the width of a DNA molecule so that the movable plate (300) remains movable relative to the nucleotide chains (106X).

### EM Signal Generation and Detection

When the sequencer apparatus (100) is configured to measure the current variations due to the electrical resistance of the sample portion (by quantum tunnelling resistance) from the reference electrode (102A) or the sample electrode (104A) adjacent the sample portion, the anchor plate (200) includes at least one electrical current source configured to provide electrical current to the reference electrode (102A)—and to the array of the reference electrodes (102Xx) or the reference electrode plate, e.g., including at least one wire/nanowire embedded in the substrate of the anchor plate (200), wherein the wire/nanowire is connected to an electrical source, e.g., a battery, that is external to the anchor plate (200), through an electrical circuit.

Each sample electrode (104Xx) may be configured for measuring the sample EM signal from the selected portion (“first portion”) of the nucleotide chain (106X) that is directly adjacent, or substantially directly adjacent, the sample electrode (104Xx). As the sample electrode (104Xx) is moved in order to modulate the sample EM signal, a different selected portion (“second portion”) of the nucleotide chain (106X) becomes (substantially) directly adjacent the sample electrode (104Xx), and the different portion has a different electronic charge and/or electromagnetic conductivity/absorption from the initial portion, and thus the sample EM signal is modulated by this difference—and since this difference depends on the difference in nucleotide bases/base-pairs that have been moved from and to sample electrode (104Xx), the detectable EM signal is indicative of the nucleotide sequence that lies between the first portion and the second portion. By calculation/processing of the sample EM signals from the plurality of the sample electrodes (104Xx), at least some of the nucleotide bases/base-pairs can be estimated/detected from the nucleotide chain (106X).

The or each reference electrode (102Xx) may be configured for measuring the reference EM signal from the reference portion of the nucleotide chain (106X) that is directly adjacent, or substantially directly adjacent, the reference electrode (102Xx). As the reference electrode (102Xx) is not moved during the measurement process, the reference EM signal should not change substantially during the movement of the sample electrodes (104Xx), and thus the reference EM signal may be substantially steady, and may provide a reference for use in de-noising processes. For the reference plate electrode, the reference EM signal includes signals from the plurality of the portions of the nucleotide chain (106X) that are directly adjacent, or substantially directly adjacent, the reference electrode (102X).

The modulation mechanism may be configured to move each sample electrode (104Xx) along the linear array of the reference electrodes (102X) by a distance at least equal to the length (in the movement direction) of each sample electrode (104Xx) plus the length of the spacing between the sample electrode (104Xx), in other words by a distance at least equal to the period of the sample electrodes (104Xx), such that each portion of the nucleotide chain (106X) under the sample electrodes (104Xx) is traversed along its length.

The length of the sample electrodes (104Xx) in the movement direction may be substantially 2 to 50 nm. Each sample electrode (104Xx) may generate its EM signal from a selected portion containing substantially 1 to 150 base-pairs if each nucleotide unit extends substantially 0.33 nm along the nucleotide chain in the movement direction.

Preferably and optionally, the sample electrodes (104Xx) may be arranged such that there is an overlap in detection ranges between different or neighbouring sample electrodes (104Xx), where the redundancy may be used for accounting for systematic variations. For example, differences in shape, or local distribution of impurities, may render one sample electrode less sensitive overall, or just to particular bases/charge densities. Overlaps in the detection range between different or neighbouring sample electrodes (104Xx) may allow reducing or eliminating one or more of these systematic variations, thereby improving the accuracy of nucleotide sequencing.

The modulation mechanism may move the sample electrodes (104Xx) at a selected frequency/periodicity such that the sample EM signals can be demodulated at a corresponding frequency to improve the signal-to-noise ratio (SNR) of the sample EM signals.

The modulation mechanism may move the sample electrodes (104Xx) based on signal feedback from the electrodes.

### Process

In operation, the system performs a process that includes:
a) holding/securing the nucleotide chain (106X) by way of the anchor molecule (118X);
b) straightening/stretching/extending/unwinding the nucleotide chain (106X) by operation of the attractor electrode (404,504)—with the actuated straightener apparatus (400) and/or the dual-electrode straightener apparatus (500);
c) the reference electrodes (102Xx), which may include the reference electrode plate, receiving the nucleotide chains (106X);
d) straightening/stretching/extending/unwinding the nucleotide chain (106X) by operation of the reference electrodes (102Xx) along each linear array;
e) the reference electrodes (102Xx) holding/securing the nucleotide chain (106X) over/along the linear array of the reference electrodes (102Xx) by electrostatic attraction between the reference electrodes (102Xx) and the nucleotide chain (106X) because the reference electrodes (102Xx) are charged (e.g., by the current source, or by semiconductor doping) to attract the nucleotide chain (106X);
f) draining the electrolyte solution;
g) moving the straightened/stretched/extended/unwound nucleotide chain (106X) from the actuated straightener apparatus (400) or the dual-electrode straightener apparatus (500) to the sequencer apparatus (100);
h) moving the anchor plate (200) and the movable plate (300) into their alignment, i.e., their operational condition, by controlling the sensor actuator (108) and/or the anchor plate actuator (124); and
i) after draining the electrolyte solution, sequencing the anchored and extended nucleotide chain (106X) by performing the measurement process in which the EM signals are detected.

The measurement process includes:
a) measuring EM signals from a sample portion of the nucleotide chain (106X); and
b) moving the nucleotide chain (106X) in order to modulate the EM signals.

The measurement process may include:
a) measuring variations in electrical current (conductance) between a reference electrode (102X) and a corresponding sample electrode (104X), caused by the effect of different local charge densities for different nucleotides;
b) measuring variations in electrical current conducted through part of the nucleotide chain (106A), from the anchor electrode (112X) to one of the sample electrodes (104Xx);
c) measuring reference electrical current conducted through part of the nucleotide chain (106A), from the anchor electrode (112X) to one of the reference electrodes (102Xx); and/or
d) measuring variations in electrical current as in (a) and (b), relative to a fixed-frequency modulation applied to the anchor plate (200) via the plate actuator (124).

### Implementations

The movable plate (300) may be formed as a silicon chip/plate, and the sample electrodes (104Xx) may be formed using a photolithography process. The sample electrodes (104Xx) may be formed as electrodes in a square 2D grid located on the surface of the silicon chip/plate top side. The movable plate (300) may include a mixed signal integrated circuit, e.g., integrated into the substrate in the underside of the silicon chip/plate, which amplifies, filters and converts analog signals from the sample electrodes (104Xx) into amplified signals and optionally digital signals for electronic demodulation/processing in an external computer.

The anchor plate (200) may be formed as a silicon chip/plate, and the reference electrodes (102Xx) may be formed using a photolithography process. The reference electrodes (102Xx) may be formed as electrodes in a square 2D grid located on the surface of the silicon chip/plate top side. The anchor plate (200) may include a mixed signal integrated circuit, e.g., integrated into the substrate in the underside of the silicon chip/plate, which amplifies, filters and converts analog signals from the reference electrodes (102Xx) into amplified signals and optionally digital signals for electronic demodulation/processing in the external computer.

Alternatively, other suitable fabrication methods/processes, e.g., guided growth, may be used for forming the sample electrodes (104Xx) on the movable plate (300) and/or forming the reference electrodes (102Xx) on the anchor plate (200).

The electrodes of the sample electrodes (104Xx) and the reference electrodes (102Xx) may be field-effect sensors. The sample electrodes (104Xx) and the reference electrodes (102Xx) are configured for measuring EM signals, as described in further detail hereinbefore.

The electrodes of the sample electrodes (104Xx) and the reference electrodes (102Xx) may be nano fabricated on the silicon wafer using the photolithography process, or any other suitable fabrication methods/processes, e.g., guided growth.

Each sample electrode (104Xx) may be made of highly p-doped silicon, and may include a thin 1-2 nm oxide layer, or a sub 1-nm oxide layer, chemically grown by natural oxidation on the surface to act as the electrical insulator between the sample electrode (104Xx) and the sample portion of the nucleotide chain (106). The sample electrodes (104Xx) may be rectangular prisms, each with a 1 to 50 nm length and 1 to 50 nm width, or a 2 to 50 nm length and 2 to 50 nm width, the length and width of each may be substantially equal (thus forming a square cross-section). Each sample electrode (104Xx) may be vertically embedded on the silicon substrate achieving a quasi-atomically flat surface throughout the entire chip, i.e., in the plane facing the nucleotide chain (106). The sample electrodes (104Xx) are separated from each other by a selected distance on all sides to mitigate electrical interference and potential quantum tunnelling effects. The selected distance (mutual spacing, or mutual separation distance) between adjacent ones of the sample electrodes (104Xx) may be substantially equal to the length or width of the sample electrodes (104Xx), e.g., each sample electrode (104Xx) may have a substantially square extent in a plane parallel to the 2D array, and inter-electrode spacing substantially equal to the sides of each square in that plane. For example: e.g., if the electrode is 1 by 1 nm, the distance or gap between electrodes can be 1 nm; or if the electrode is 2 by 2 nm, the distance or gap between electrodes can be 2 nm. The inner side of each sample electrode (104Xx)—facing away from the nucleotide chain (106)—may connect to a gate of a CMOS transistor that acts as a signal amplifier.

In the first embodiment, each reference electrode (102Xx) may be made of highly p-doped silicon, and may include a thin 1-2 nm oxide layer, or a sub 1-nm oxide layer, chemically grown by natural oxidation on the surface to act as the electrical insulator between the reference electrode (102Xx) and the sample portion of the nucleotide chain (106). The reference electrodes (102x) may be rectangular prisms. The reference electrodes (102Xx) may each have a 1 to 50 nm length and 1 to 50 nm width, or a 2 to 50 nm length and 2 to 50 nm width, the length and width of each may be substantially equal (thus forming a square cross-section), and the reference electrodes (102Xx) may be referred to as including “nano tips”, “nano tip electrodes” or “probes” having these lengths and widths. Each reference electrode (102Xx) may be vertically embedded on the silicon substrate achieving a quasi-atomically flat surface throughout the entire chip, i.e., in the plane facing the nucleotide chain (106). The reference electrodes (102Xx) are separated from each other (i.e., mutually separated) by a selected distance on all sides to mitigate electrical interference and potential quantum tunnelling effects between neighbouring directly-adjacent electrodes. The selected distance (mutual spacing, or mutual separation distance) may be equivalent to their length or width (or substantially equal to their length or width), e.g., if the electrode is 2 by 2 nm the distance between electrodes can be selected to be 2 nm. The inner side of each reference electrode (102Xx)—facing away from the nucleotide chain (106)—may connect to a gate of a CMOS transistor that acts as a signal amplifier. Alternatively, the inner side of each reference electrode (102Xx) may electrically communicate via an electronic circuit, including the mixed signal integrated circuit, to the external computer.

The anchor electrodes (112X) may be formed of n-doped silicon, and the anchor molecules (118X) are thus selected/configured to bond with the n-doped silicon. The anchor electrodes (112X) may be connected/configured to conduct electrical current through to the anchor molecule (118X) and thus to the nucleotide chain (106A)

The general support structure (116) may include a dense block of material, e.g., granite, suspended on an air table to mitigate external vibrations during the operation of the sequencer apparatus (100). The general support structure (116) may include an EM insulating cage/shell (referred to as a “Faraday cage”) surrounding the sample portion and the anchor portion to mitigate EM interference when the sequencer apparatus (100) is operating: the cage/shell may include a metallic alloy of Titanium Carbonitride.

### III. Second Embodiment

A second embodiment of the system including the apparatus 100 is described hereinafter.

### General Structure of the Sequencing Apparatus

As shown in FIG. 6, the sequencer apparatus (100) includes:
a) at least one electromagnetic (EM) sample electrode (“sample electrode” 104A, including 104Aa, 104Ab, . . . , 104An) for measuring an EM signal (“sample EM signal”) (e.g., an electrical charge and/or an electrical current) from a portion (or “sample portion”) of a nucleotide chain (106A); and
b) a mechanism (or “modulation mechanism”) configured to move the nucleotide chain (106A) relative to the EM sample electrode (104A), in order to modulate the EM signal.

The at least one sample electrode (104A) may be a non-contact EM sensor. That is, it does not contact or touch the nucleotide chain (106A) during the measuring process. The sample electrode (104A) may take the form of, e.g., a nanotip electrode, which may be referred to hereinafter as “nanotip” or “probe”.

The at least one sample electrode (104A) is provided in a sensor portion of the apparatus (100). The sensor portion includes:
a) a sensor plate (700) with an array of one or more of the sample electrodes (104Aa, 104Ab, . . . , 104An); and
b) a sensor support structure (722) that fixes the sensor plate (700) to a general support structure (116).

The mechanism includes an actuator configured to move a shared collection plate (600) on which the nucleotide chain (106A) is deposited.

More specifically, the apparatus (100) has a collection plate portion, including:
a) the shared collection plate (600), being a conductive baseplate for holding/supporting the nucleotide chain (106A);
b) a collection plate actuator (624) for moving the shared collection plate (600); and
c) a plate support structure (614) that connects the shared collection plate (600), including via the collection plate actuator (624), in controllably movable relation to a general support structure (116) during a measurement process of the sequencer apparatus (100).

As shown in FIG. 7, the sensor plate (700) may include one or more arrays of the two or more sample electrodes (104An), thus forming a two-dimensional (2D) array of the sample electrodes (104Aa, 104Ab, . . . , 104An; 104Ba, 104Bb, . . . , 104Bn; 104Na, 104Nb, . . . , 104Nn), the tip of each electrode facing the shared collection plate (600). Each of the arrays in the 2D array may be substantially linear in the sense of forming a line or chain or series of adjacent electrodes, each electrode being adjacent the next one in the series. The 2D array may be formed of multiple ones of these line arrays (which may be one dimensional, or may have some bends as long as the electrodes substantially form the line or chain or series), and these line arrays may be mutually adjacent or next to each other, including being parallel, or substantially parallel to each other (e.g., with corresponding bends), or at least substantially mutually adjacent, thus forming the 2D array.

When the nucleotide chain (106A) is deposited on the shared collection plate (600), it is arranged and secured in position (e.g., by Van der Waals forces) such that the nucleotide chain (106A) is aligned linearly under/along the corresponding linear array of the sample electrodes (104a, 104a, . . . , 104n). Each sample electrode (104A) addresses a plurality of nucleotides.

The EM signals measured by the sample electrode (104A) may include one or more of the following: an electrical charge, an electrical current and/or a voltage.

The measured EM signals indicate electromagnetic variations due to quantum tunnelling. Quantum tunnelling is a quantum mechanical phenomenon where under certain conditions (e.g., distance, bias voltage) electrons have a finite probability to cross the potential barrier determined by the physical gap between the electrodes.

Due to quantum tunnelling, when in the proximity of the sample electrode (104A), the DNA molecule perturbs the conditions set, thus creating a measurable electromagnetic signal arising from the difference in electron flow. The perturbation of the tunnelling process has repeatable characteristics (or “signature”) that can be used to differentiate nucleotides of different types (e.g., by using algorithms trained with the EM characteristics or signature for each type of nucleotide), therefore allowing sequencing of the nucleic chain. For instance, signal characteristics/signatures can be extracted from the measured EM signals (e.g., in the form of current-voltage (I-V) curves, current-time (I-t) traces, and/or other processed signals derived from these measurements).

In the embodiment described herein, the relative movement between the nucleotide chain (106A) and the EM sample electrode (104A) is achieved by moving the nucleotide chain (106A), and more specifically, by moving the shared collection plate (600) where the nucleotide chain (106A) is deposited. Alternatively, this relative movement may be achieved by moving the sample electrode (104A) instead of, or in addition to, the shared collection plate (600). For example, a sensor actuator (118) as described in the first embodiment may be provided to enable movement of the sensor plate (700), where the sample electrode (104A) is provided. However, moving the shared collection plate (600) rather than the sensor plate (700) may allow reducing mechanical stress imposed on the sensor plate (700), and avoiding vibration of the sample electrode (104A) that may be caused by the movement, thereby achieving more accurate measuring results and sequencing results.

### Straightening Apparatuses

The system may include at least one apparatus (“straightening apparatus”) for straightening/stretching/extending/unwinding the nucleotide chains (106A, 106B, . . . , 106N) before insertion into the sequencer apparatus (100). The straightening apparatus may have the same or similar structure as described hereinabove in the first embodiment.

Alternatively, the straightening of the nucleotide chains (106A, 106B, . . . , 106N) may be performed using molecular threading technology, e.g., as described in Molecular threading: mechanical extraction, stretching and placement of DNA molecules from a liquid-air interface. Payne, Andrew C., et al. PloS one 8.7 (2013): e69058.

### Actuators and Alignment

The collection plate actuator (624) may include piezo-electric actuators, e.g., from PI (Physik Instrumente), or piezoceramic actuators.

The collection plate portion may include a stepper motor, which is configured to move the shared collection plate (600) into alignment (or “position” or “operational condition”) with the sensor plate (700)—including in all 3 orthogonal directions, X, Y, Z, and additionally/optionally three rotational dimensions (e.g. Euler angles α, β, γ)—after the nucleotide chains (106X) have been deposited in place on the shared collection plate (600). There is a small gap between the plates (600,700) and they are substantially plane parallel, e.g., the small gap may be less than 1 micrometer, less than 100 nanometers (nm), or less than 10 nm, e.g., of the order of a width of a DNA molecule (substantially 0.3 nm to 2.5 nm), but substantially more than the width of a DNA molecule so that the nucleotide chains (106X) and the shared collection plate (600) remain controllably movable relative to the sensor plate (700).

### EM Signal Generation and Detection

When the sequencer apparatus (100) is configured to measure the sample EM signal (e.g., electrical charge, voltage and/or current) from the sample electrode (104A) adjacent to the sample portion, the sensor plate (700) includes at least one electrical current source configured to provide respective electrical voltages/currents to the sample electrodes (104Xx), e.g., at least one wire/nanowire embedded in the substrate of the sensor plate (700) is connected to an electrical power source, e.g., a battery, that is external to the sensor plate (700), through an electrical circuit. On the other side, the shared collection plate (600), being a conductive plate, is electrically connected to the opposite electrode of the electrical source, directly or through an electrical circuit.

Each sample electrode (104Xx) is configured for measuring the sample EM signal from the selected portion (“first portion”) of the nucleotide chain (106X) that is directly adjacent, or substantially directly adjacent, the sample electrode (104Xx).

The selected portion (“first portion”) of the nucleotide chain (106X) may include a plurality of nucleotides. Accordingly, the measured sample EM signal is a collective value associated with all of these nucleotides in the sample portion. To identify the EM signal associated with each individual nucleotide, the sample EM signal is modulated.

The modulation of the sample EM signal may be performed by moving (e.g. dithering) the shared collection plate (600), such that the nucleotide chain (106X) is moved in the longitudinal direction of the nucleotide chain (106X), causing a relative movement between the nucleotide chain (106X) and the sample electrode (104Xx).

As a result of the movement, a different selected portion (“second portion”) of the nucleotide chain (106X) becomes directly or substantially adjacent to the sample electrode (104Xx), and the different portion has a different electronic charge, current and/or voltage from the initial portion (first portion), and thus the change in the sample EM signal reflects this difference—and since this difference depends on the difference in nucleotide bases/base-pairs that have been moved from and to sample electrode (104Xx), the detectable EM signal is indicative of the nucleotide sequence that lies between the first portion and the second portion. By calculation/processing of the sample EM signals from the plurality of the sample electrodes (104Xx), at least some of the nucleotide bases/base-pairs can be estimated/detected from the nucleotide chain (106X).

The modulation mechanism may be configured to move the shared collection plate (600) in the longitudinal direction of the nucleotide chain (106X) by a distance at least equal to the length (in the movement direction) of each sample electrode (104Xx) plus the length of the spacing between the sample electrode (104Xx), in other words by a distance at least equal to the period of the sample electrodes (104Xx), such that each portion of the nucleotide chain (106X) under the sample electrodes (104Xx) is traversed along its length.

The length of the sample electrodes (104Xx) in the movement direction may be substantially 2 to 50 nm. Each sample electrode (104Xx) may generate its EM signal from a selected portion containing substantially 1 to 150 base-pairs if each nucleotide unit extends substantially 0.33 nm along the nucleotide chain in the movement direction.

Preferably and optionally, the sample electrodes (104Xx) may be arranged such that there is an overlap in detection ranges between different or neighbouring sample electrodes (104Xx), where the redundancy may be used for accounting for systematic variations. For example, differences in shape, or local distribution of impurities, may render one sample electrode less sensitive overall, or just to particular bases/charge densities. Overlaps in detection ranges between different or neighbouring sample electrodes (104Xx) may allow reducing or eliminating one or more of these systematic variations, thereby improving the accuracy of nucleotide sequencing.

The modulation mechanism may move the shared collection plate (600) at a selected frequency/periodicity such that the sample EM signals can be demodulated at a corresponding frequency to improve the signal-to-noise ratio (SNR) of the sample EM signals.

The movement of the shared collection plate (600) may be driven by, for example, one or more standard off-the-shelf drivers, configured to attain suitable modulation frequencies. The frequency of the modulation may be selected based on the spectral composition of the noise. For example, it may be selected to encode the signal into a frequency where the noise level is the lowest. Most noise is expected to be in the lowest frequency region (up to tens of kHz), which may be mitigated by a solid support base (e.g. granite base) and a vibration isolation table. Preferably, the modulation mechanism may allow changing the modulation frequency. This may allow flexibility in choosing the modulation frequency that delivers the best noise-reducing effect, and may ensure robustness from possible noise sources. In examples, the modulation frequency from the modulation mechanism may be in the range 1 Hz to 100 Hz, and may be variable between two selected frequencies in the range 1 Hz to 100 Hz, e.g., between 1 Hz and 100 Hz.

Optionally, in addition to longitudinal modulation, the shared collection plate (600) may be dithered vertically by way of a vertical modulation. The vertical modulation may have a vertical modulation frequency in the range 1 Hz to 100 Hz, optionally variable between two selected frequencies in the range 1 Hz to 100 Hz, e.g., between 1 Hz and 100 Hz. The vertical modulation, if present, may be independent from the horizontal modulation mechanism.

Alternatively, or additionally, the modulation of the sample may be performed by changing an input electromagnetic signal to the sample electrodes (104Xx), including by electrically modulating the respective electrical voltages/currents being provided to the sample electrodes (104Xx), which can cause a change in the measured sample EM signal from each sample electrode (104Xx), referred to as “electrical modulation”. The electrical modulation of the input electrical voltages/currents may be conducted by, for instance, standard off-the-shelf electronics/software (e.g., off-the-shelf lock-in amplifiers). The electrical modulation frequency may be selected to reduce or mitigate noise, including noise arising from in-circuit electronics (e.g., based on the spectral composition of the noise). For instance, it may be selected to encode the signal into a frequency where the noise level is the lowest. The electrical modulation may include an electrical modulation frequency in the range 1 Hz to 100 Hz, optionally variable between two selected frequencies in the range 1 Hz to 100 Hz, e.g., between 1 Hz and 100 Hz. The EM modulation, if present, may be independent from the vertical modulation and the horizontal modulation.

By calculation/processing of the sample EM signals from the plurality of the sample electrodes (104Xx) based on the input electromagnetic signal, at least some of the nucleotide bases/base-pairs can be estimated/detected from the nucleotide chain (106X).

### Process

In operation, the system according to this embodiment performs a process that includes:
a) depositing the nucleotide chain (106X) on the shared collection plate (600);
b) moving the shared collection plate (600) and the sensor plate (700) into their alignment, i.e., their operational condition, by controlling the collection plate actuator (624); and
c) sequencing the nucleotide chain (106X) on the shared collection plate (600) by performing the measurement process in which the EM signals are detected.

The measurement process includes:
a) measuring EM signals from a sample portion of the nucleotide chain (106X) using the sample electrodes (104X); and
b) moving the nucleotide chain (106X) relative to the sample electrodes (104X) in order to modulate the EM signals.

The measured EM signal includes one or more of the following: an electrical charge, an electrical current, and a voltage.

Preferably, the nucleotide chain (106X) is straightened/stretched/extended/unwound before being moved to the shared collection plate (600). The straightening/stretching/extending/unwinding process may be performed by using molecular threading technology. Alternatively, it may be performed by using a straightener apparatus, e.g., the actuated straightener apparatus (400) or the dual-electrode straightener apparatus (500) as described hereinabove in the first embodiment. The straightener apparatus may be integrated into or separate from the system of the present disclosure. Alternatively, the straightening/stretching/extending/unwinding process may be performed by using any other nucleotide chain straightening techniques/apparatuses applicable to the described embodiment.

The measured EM signals are collective values associated with all of the nucleotides in the sample portion of the nucleotide chain (106X). To identify the EM signal associated with each individual nucleotide, the sample EM signal is demodulated based on a movement signal associated with the movement of the nucleotide chain (106X) relative to the sample electrodes (104X). The movement signal may include a signal (which may be referred to as a “dither signal”) indicating the distance and timing of the relative movement of the nucleotide chain relative (106X) to the at least one sample electrode (104X) in the longitudinal direction of the nucleotide chain (106X). The modulation/demodulation process may also allow isolating the sequencing information from other forms of noise.

From the demodulated EM signals (e.g., in the form of current-voltage (I-V) curves, and/or current-time (I-t) traces), unique characteristics or signatures associated with each type of known nucleotides can be extracted. The extracted signal characteristics or signatures are then used for resolving/identifying the type of nucleotide in the sample portion of the nucleotide chain (106X), which can be performed using a database or an automatic classifier (e.g., a proprietary algorithm trained with EM characteristics/signatures for each type of nucleotides).

Optionally or alternatively, the sample EM signals may be modulated by controlling/changing an input EM signal to one or more of the sample electrodes (104X), e.g., the electrical current or voltage applied to one or more of the sample electrodes (104X). This may allow providing more extractable signal characteristics or signatures for resolving/identifying the type of each nucleotide in the sample portion of the nucleotide chain (106X), and thus may improve the accuracy and efficiency of sequencing.

### Implementations

The sensor plate (700) may be formed as a silicon chip/plate, and the sample electrodes (104Xx) may be formed using a photolithography process. The sample electrodes (104Xx) may be formed as electrodes in a square 2D grid located on the surface of the silicon chip/plate top side. Alternatively, other suitable fabrication methods/processes, e.g., guided growth, may be used for forming the sample electrodes (104Xx) on the sensor plate (700). The sensor plate (700) may include a mixed signal integrated circuit, e.g., integrated into the substrate in the silicon chip/plate, which amplifies, filters and converts analog signals from the sample electrodes (104Xx) into amplified signals and optionally digital signals for electronic demodulation/processing in an external computer.

The shared collection plate (600) may be formed as a conductive silicon chip/plate. The shared collection plate (600) may be connected to, via the collection plate actuator (624), the same external computer for controlling/processing the signals to/from the sensor plate (700).

The system includes an electronic system (800). As shown in the schematic diagram of FIG. 8, an external computer (“PC control (802)”) is electrically connected to the shared collection plate (600) via the collection plate actuator taking the form of a piezo 3D stage (804). The sample electrodes (104Xx), in the form of a multi-electrode array plate (818), are controlled by the external computer (PC control (802)) via a multi-digital switch (816). The EM signals collected by the multi-electrode array plate (818) are processed by a low noise amplifier (814) and a lock-in current amplifier (812), which is synchronised to the movement signal (“dither signal”), and converted to digital data by an analog-digital converter (“ADC (810)”), which is then collected by a data collect FPGA (808) and sent to the PC control (802). The data collect FPGA (808) also collects the data sent from the PC control (802) to the multi-digital switch (816) for controlling the input signals to the multi-electrode array plate (818).

The sample electrodes (104Xx) are configured to emit/generate EM fields/charge or electrons (for the quantum tunnelling).

Using photolithography processes, the sample electrodes (104Xx) may be nano-fabricated on the silicon wafer. Each sample electrode (104Xx) may be made of highly p-doped silicon, and may include a thin 1-2 nm oxide layer, or a sub 1-nm oxide layer, chemically grown by natural oxidation on the surface to act as the electrical insulator between the sample electrode (104Xx) and the sample portion of the nucleotide chain (106X). The sample electrodes (104Xx) may be rectangular prisms, each with a 1 to 50 nm length and 1 to 50 nm width, or a 2 to 50 nm length and 2 to 50 nm width, the length and width of each may be substantially equal (thus forming a square cross-section), and the sample electrodes (104Xx) having these lengths and widths may be referred to as “nano tips”, “nano tip electrodes” or “probes”. Each sample electrode (104Xx) may be vertically embedded on the silicon substrate achieving a quasi-atomically flat surface throughout the entire chip, i.e., in the plane facing the nucleotide chain (106). The sample electrodes (104Xx) are separated from each other (i.e., mutually separated) by a selected distance on all sides to mitigate electrical interference and potential quantum tunnelling effects between neighbouring directly-adjacent electrodes. The selected distance (mutual spacing, or mutual separation distance) may be equivalent to their length or width (or substantially equal to their length or width), e.g., if the electrode is 2 by 2 nm the distance between electrodes can be selected to be 2 nm. The inner side of each sample electrode (104Xx)—facing away from the nucleotide chain (106X)—may connect to a gate of a CMOS transistor that acts as a signal amplifier. Alternatively, the inner side of each sample electrode (104Xx) may electrically communicate via an electronic circuit, including the mixed signal integrated circuit, to the external computer.

The general support structure (116) may include a dense block of material, e.g., granite, suspended on an air table to mitigate external vibrations during the operation of the sequencer apparatus (100). The general support structure (116) may include an EM insulating cage/shell (referred to as a “Faraday cage”) surrounding the sample portion and the collection plate portion to mitigate EM interference when the sequencer apparatus (100) is operating: the cage/shell may include a metallic alloy of Titanium Carbonitride.

### IV. Applications

In the genetic sequencing industry, there may be major shortfalls regarding length, time, accuracy, reliability, scalability and price. There may be many devices in the industry each with its merits and downfalls, therefore scientists are forced to use multiple devices/products and techniques to sequence their samples depending on their needs and what they are willing to sacrifice (time, accuracy, etc.). These issues have led to the industry not achieving its full potential and thus humanity not benefiting from it. The system and apparatus and process described herein may address these issues.

Regarding length and time, due to the highly parallel nature of the sequencer apparatus (100) and electromagnetic basis of the sample and reference electrodes (104,102), massively parallel “strands”/“strings” of DNA, ssDNA or RNA can be analysed with high uniformity. As a genome may be arranged in a 2D sheet in the sequencer apparatus (100), the electrode arrays (104X, 102X) can be formed using 2D fabrication techniques and equipment, e.g., from the semiconductor industry, e.g., using a grid of electrically conductive electrodes. By creating oscillations by the actuators (including the anchor plate actuator (124) and/or the sensor actuator (108), e.g., piezoceramic actuators), variances in the electromagnetic signals (e.g. electrical charge, voltage or current) are detectable via the electronic circuitry. Since an entire genome may be sequenced at the same time, this may reduce sequencing time from days to minutes. Due to micro-oscillations in the relative movement of the sample electrodes (104Xx) and the nucleotide chains (106X), and the small dimensions of the sample and reference electrodes (104,102), single base accuracy may be achievable. The sequencer apparatus (100) need not become clogged during the sequencing process, and may be easily cleaned and reused rapidly, potentially allowing the sequencing of multiple whole human genomes per day with one sequencer apparatus (100). As no polymerase chain reaction (PCR) amplification is required, the use of the system may be substantially less costly than previous technologies.

### V. Interpretation

The FIGs. included herewith show aspects of non-limiting representative embodiments in accordance with the present disclosure, and particular structural elements shown in the FIGs. may not be shown to scale or precisely to scale relative to each other. The depiction of a given element or consideration or use of a particular element number in a particular FIG. or a reference thereto in corresponding descriptive material can encompass the same, an equivalent, an analogous, categorically analogous, or similar element or element number identified in another FIG. or descriptive material associated therewith. The presence of “/” in a FIG. or text herein is understood to mean “and/or”, i.e., “X/Y” is to mean “X” or “Y” or “both X and Y”, unless otherwise indicated. The recitation of a particular numerical value or value range herein is understood to include or be a recitation of an approximate numerical value or value range, for instance, within +/−20%, +/−15%, +/−10%, +/−5%, +/−2.5%, +/−2%, +/−1%, +/−0.5%, or +/−0%. The term “essentially all” or “substantially” can indicate a percentage greater than or equal to 50%, 60%, 70%, 80%, or 90%, for instance, 92.5%, 95%, 97.5%, 99%, or 100%.

Many modifications will be apparent to those skilled in the art without departing from the scope of the present invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

### VI. Exemplary Embodiments

Exemplary embodiments provide direct sequencing of the nucleotide chains (or “nucleic chains”) via direct (or “first-hand”) real-time measurements of the electronic configuration along the molecule using the 2D array (also referred to as the “electrode matrix/grid”) of the sample electrodes. In some exemplary embodiments, the sample electrodes have substantially square cross-sections that taper towards their free ends, e.g., forming a curved tip which may be easier to fabricate than a square-topped tip. The sample electrodes may be regarded as similar to scanning tunnelling microscope (STM) tips, but each sample electrode is not an atomic-scale nanotip (as in an STM) but a larger nanotip that is easier to fabricate in an array using standard commercially available photolithography fabrication tools and processes. Each sample electrode may operate to provide electronic characteristics of the plurality of nucleotides (or “group”) probed by the sample electrode by measuring the current-voltage (I-V) profiles in a similar method to previous STM-based methods for scanning DNA molecules with single ultra-sharp nanoscale conductive tips, e.g., as described in Shapir, E. (2007), ‘Electronic structure of single DNA molecules resolved by transverse scanning tunnelling spectroscopy’, Nature Materials, 7, 68. 10.1038/nmat2060. In some examples, the system may be configured to detect a tunnelling current at each sample electrode from substantially 2 pA (pico Amps), up to substantially 10 nA (nano Amps), e.g., substantially 10-100 pA with a fixed bias under 10V. In some examples, while the small gap between the plates is substantially equal to, whilst being greater, than the width of a DNA molecule (substantially 0.3 nm to 3 nm), the gap from each sample to the nucleotide chain, in use, can be substantially equal to the width of a DNA molecule, e.g., substantially 1 nm or 3 nm. In examples, the measured EM signals may include: current over time (I-t) with a fixed bias voltage; current against distance (I-z) with at a fixed bias voltage and the sample electrode travelling along a direction orthogonal to the substrate to approach the molecule in a repeatable fashion and gather tunnelling data (I) as a function of distance (z); current-voltage curves (I-V), and derivatives thereof, with the tunnelling current being recorded as a function of a varying bias voltage, e.g., using analysis techniques described in Tanaka, H. (2009), ‘Partial sequencing of a single DNA molecule with a scanning tunnelling microscope’, Nature Nanotechnology, 4, 518. 10.1038/NNANO.2009.155, and/or Tanaka, H. (2017), ‘Sequencing of adenine in DNA by scanning tunneling microscopy’, Japanese Journal of Applied Physics, 56, 08LB02. 10.7567/JJAP.56.08LB02. The EM signals may be processed to identify the nucleotides using deterministic signal processing analysis and software as well as machine learning techniques. The process may include the use of Density Functional Theory (DFT) to provide results as data points to assist identification. The process may include transverse measurements as described in Zwolak, M. (2005), ‘Electronic Signature of DNA Nucleotides via Transverse Transport’, Nano Letters, 5, 421. 10.1021/nl048289w. Some embodiments may use machine learning methods to identify DNA molecules, e.g., as described in Albrecht, T. (2017), ‘Deep learning for single-molecule science’, Nanotechnology, 28, 423001. 10.1088/1361-6528/aa8334, or Im, J. (2018), ‘Recognition Tunneling of Canonical and Modified RNA Nucleotides for Their Identification with the Aid of Machine Learning’, ACS Nano, 12, 7067. 10.1021/acsnano.8b02819. An exemplary embodiment may have the 2D array formed by a 10×10 electrode array, with printed direct contacts for each electrode to external circuitry, and connectors integrated and covered with an insulating material (e.g., SiO2). The movement mechanism may be provided by a commercially available 6-axis piezoelectric system. The movement mechanism is also responsible for mechanically modulating the shared collection plate so that the DNA strands are oscillated relative to the measurement electrode. This modulation may be used to encode signal data (representative of the molecules being measured) via the process of Modulation Encoding. This signal is later decoded via use of deterministic algorithms for signal processing as well as machine learning techniques. The modulation frequency described hereinbefore may have a tuneable frequency ranging from 1 Hz to 100 Hz to better distinguish the detected EM signals (e.g., current) from noise, and the detected EM signals can thus be filtered with a corresponding electronic filter or demodulator circuit corresponding to the electronic modulation (e.g., having a corresponding frequency or frequency pattern). In some embodiments, the EM signals collected by the multi-electrode array plate (818) may be connected to the low noise amplifier (814) by an N-to-1 switch (e.g., including a commercially available multiplexer switch, e.g., from “Analog Devices”) to reduce the consumption of the amplifying modules: this may reduce the sampling speed of the system but reduce cost by multiplexing the amplifier module (e.g., by transforming the N×M parallel signals into N parallel signals with M serial signals). In some embodiments, the low noise amplifier (814) may include a commercially available picoampere input current quad operational amplifier from “Analog Devices”, the ADC (810) may include a commercially available ADC module from “Texas Instruments”, the data collect FPGA (808) may include a commercially available FPGA from “Intel”, and the lock-in current amplifier (812) may include a commercially available modulator/demodulator module from “Texas Instruments”.

1. A system for nucleotide chain sequencing, the system including an apparatus with:
at least one electrode for measuring a collective electromagnetic signal from a portion of a nucleotide chain, the portion including a plurality of nucleotides; and
a mechanism configured to move the nucleotide chain relative to the at least one electrode in order to modulate the collective electromagnetic signal, for identifying an electromagnetic signal associated with each individual nucleotide in the plurality of nucleotides.

2. The system of claim 1, wherein:
the apparatus includes a conductive plate for holding the nucleotide chain, and
the mechanism is configured to move the conductive plate relative to the at least one electrode.

3. The system of claim 1, wherein the at least one electrode includes at least one nanotip electrode.

4. The system of claim 1, wherein the at least one electrode includes a two-dimensional array of electrodes.

5. The system of claim 4, wherein:
the apparatus is configured for sequencing a plurality of nucleotide chains; and
the two-dimensional array of electrodes include a corresponding plurality of lines of electrodes, each line of electrodes is configured for measuring a corresponding one of the plurality of nucleotide chains.

6. The system of claim 1, wherein the collective electromagnetic signal includes one or more of the following: an electrical charge, an electrical current, or a voltage.

7. The system of claim 1, wherein the collective electromagnetic signal is further modulated by changing an input electromagnetic signal to the at least one electrode.

8. The system of claim 1, wherein the mechanism is configured to move the nucleotide chain relative to the at least one electrode in the longitudinal direction of the nucleotide chain to modulate the collective electromagnetic signal.

9. The system of claim 8, wherein the at least one electrode includes a plurality of electrodes, and wherein the mechanism is configured to move the nucleotide chain relative to the electrodes in the longitudinal direction of the nucleotide chain by a distance equal or greater than a sum of:
(i) a length of one of the electrodes in the longitudinal direction of the nucleotide chain; and
(ii) the distance between two adjacent ones of the electrodes in the longitudinal direction of the nucleotide chain.

10. The system of claim 1, wherein the mechanism is configured to move the nucleotide chain at a selected frequency.

11. The system of claim 10, wherein the frequency is changeable by the mechanism.

12. The system of claim 10, wherein the frequency is selected based on a spectral composition of measured noise.

13. The system of claim 1, wherein the at least one electrode includes a plurality of electrodes, the electrodes being arranged such that there is an overlap in detection ranges between two adjacent ones of the electrodes.

14. A process for nucleotide chain sequencing, including:
measuring collective electromagnetic signals from a portion of a nucleotide chain using at least one electrode, the portion of the nucleotide chain including a plurality of nucleotides; and
moving the nucleotide chain relative to the at least one electrode in order to modulate the collective electromagnetic signals, for identifying an electromagnetic signal associated with each individual nucleotide in the plurality of nucleotides.

15. The process of claim 14, further including demodulating the measured collective electromagnetic signals based on a movement signal associated with the movement of the nucleotide chain relative to the at least one electrode.

16. The process of claim 15, wherein the movement signal includes a signal associated with the movement of the nucleotide chain relative to the at least one electrode in the longitudinal direction of the nucleotide chain.

17. The process of claim 14, wherein the nucleotide chain is moved relative to the electrodes in the longitudinal direction of the nucleotide chain by a distance equal or greater than a sum of:
(i) a length of one of the electrodes in the longitudinal direction of the nucleotide chain; and
(ii) the distance between two adjacent ones of the electrodes in the longitudinal direction of the nucleotide chain.

18. The process of claim 14, wherein the nucleotide chain is moved at a selected frequency.

19. The process of claim 18, wherein the frequency is selected based on a spectral composition of measured noise.

20. The process of claim 14, wherein the collective electromagnetic signal includes one or more of the following: an electrical charge, an electrical current, or a voltage.

21. The process of claim 14, further including changing an input electromagnetic signal to the at least one electrode to modulate the measured collective electromagnetic signals.
